SEQUENCE LISTING (7 sequences)

SEQ ID NO: 1 - DNA, 20 bp, Artificial sequence
  Synthetic sequence Primer FDS EX2-3 U
cctccttggggcgtaactca

SEQ ID NO: 2 - DNA, 20 bp, Artificial sequence
  Synthetic sequence Primer FDS EX2-3 L
gccacaggtgaatgccacac

SEQ ID NO: 3 - DNA, 19 bp, Artificial sequence
  Synthetic sequence Primer FDS EX2-3 S1
ttttgttccctgcgtatcc

SEQ ID NO: 4 - DNA, 22 bp, Artificial sequence
  Synthetic sequence Primer FDS-EX12-U
ggagtagaggatgcctggtatg

SEQ ID NO: 5 - DNA, 26 bp, Artificial sequence
  Synthetic sequence Primer FDS-EX12-L
aggttacacgattatttattgagagc

SEQ ID NO: 6 - DNA, 18 bp, Artificial sequence
  Synthetic sequence Primer FDS-EX12-S1
gtgggtggctttggagat

SEQ ID NO: 7 - DNA, 401 bp, Homo sapiens
tggggtactttactctgtaccgcctccttacccagccttgtgcacgccatcttgaaggcactgagttctagcctgtttattgtaagtggtgattagttgggtctcagtcacccagccatacttttttgttccctgcgtatccttcctgtaattgtccccaagcacattccacaagagggaggggcactctgggctaaggckggggtgggagttatctggggagctgccaccatgcctctgcctttggtgcttgcccctgcagggagtgcttagtgccccctccctatgccactcccaggatgcccctgtcccgctggttgagatctgtgggggtcttcctgctgccagccccctactgggcaccccgggagaggtggctgggttccctacggcggccctcc

(19) United States
(12) Patent Application Publication
(10) Pub. No.: US 2010/0168067 A1
(43) Pub. Date: Jul. 1, 2010

(54) BIOMARKERS FOR BISPHOSPHONATE-RESPONSIVE BONE DISORDERS

(75) Inventors: John Chamberlain, London (GB); Halina Fitz-Clarence, London (GB); Mark Thomas, London (GB)

Correspondence Address: LAHIVE & COCKFIELD, LLP;FLOOR 30, SUITE 3000, ONE POST OFFICE SQUARE, BOSTON, MA, 02109, US

(73) Assignee: UCL BUSINESS PLC, London (GB)

(21) Appl. No.: 12/293,763

(22) PCT Filed: Mar. 19, 2007
(86) PCT No.: PCT/GB2007/000941

§ 371 Date: Sep. 19, 2008

Related U.S. Application Data
(60) Provisional application No. 60/783,834, filed on Mar. 21, 2006.

(51) Int. Cl. A61K 31/675; C12Q 1/68; A61K 31/66; A61P 19/08; A61P 19/10

(52) U.S. Cl. 514/89; 435/6; 514/94; 514/108

(57) ABSTRACT

This invention relates to the finding that the presence of polymorphisms in and around the farnesyl diphosphate synthase (FDPS) gene is predictive of the densitometric response of patients with bone disorders, such as osteoporosis, subsequent to commencing treatment with amino-bisphosphonates. Methods relating to the identification of individuals having bone disorders which are responsive to bisphosphonates and predicting the responsiveness of individuals with bone disorders to treatment with a bisphosphonate are provided.

This invention relates to biomarkers useful in predicting whether an individual having a bone disorder such as osteoporosis is likely to be responsive to treatment with bisphosphonate drugs.

Bone disorders, such as osteoporosis, result in a decrease in bone mass and bone density and/or an increased risk and/or incidence of fracture. Oral bisphosphonates are the commonest first-choice treatment where a reduction in osteoclasis would be beneficial, for example, for post-menopausal osteoporosis—a common condition affecting one third of post-menopausal women in the UK. There are estimated to be 1 million cases of osteoporosis in the UK, with 70000 hip, 120000 vertebral and 50000 wrist fractures yearly. In the US, up to 10 million patients have been suggested to be suffering from osteoporosis with around 1.5 million associated fragility fractures yearly. As the population demographic in industrialised societies ages the number of such fragility fractures is expected to increase threefold (Osteoporosis Int 1992; 2:285-289).

The total world market for drugs for treating bone disorders surpassed an estimated £2.76 billion in 2002 and is projected to exceed £6.35 billion by 2006. Bisphosphonates command the majority share of this market and are widely reimbursed on the basis of a favourable pharmaco-economic profile for fracture prevention. Yet around 40% of individuals treated with bisphosphonates do not fully respond to the drug. This represents a major value deficit both in terms of evident cost and adverse event associated morbidity (Gastro-intestinal intolerance, hypersensitivity reactions, headache, musculo-skeletal pain). Such considerations impose significant limitations on the use of such bisphosphonates in the primary care market.

The present inventors have shown that polymorphism in and around the coding region of the farnesyl diphosphate synthase (FDPS) gene is predictive of the densitometric response of patients subsequent to commencing treatment with amino-bisphosphonates.

An aspect of the inventon provides a method of identifying an individual having a bone disorder which is responsive or likely to be responsive to bisphosphonate, or predicting the responsiveness of an individual with a bone disorder to treatment with a bisphosphonate, the method comprising:
determining in a nucleic acid sample obtained from the individual, the presence or absence of a variant allele at one or more sites of polymorphism in the region of the FDPS gene,
the presence of a variant allele at the one or more sites being indicative that the individual is responsive to bisphosphonates.

Farnesyl diphosphate synthase (FDPS) (EC 2.5.1.10) catalyzes the formation of both geranyl diphosphate and isopentenyl diphosphate from diphosphate and trans,trans-farnesyl diphosphate in the isoprene biosynthetic pathway. The human FDPS protein sequence has the database entry NP_{—}001995.1 GI: 4503685. The nucleic acid sequence encoding human FDPS has the database entry NM_{—}002004.2 GI: 41281370. The human FDPS gene is located at 1q22 and has the gene reference GeneID: 2224 and the locus tags: HGNC: 3631 and MIM 134629. The sequence of the human FDPS gene is set out between bases 5769105-5780811 of the contig sequence gi|51458934 NT_{—}004487.17 and between bases 5385993-5397702 of the contig sequence gi|51460383 NT_{—}086596.1 (positions 152092649 and 152103528 on chromosome 1).

The presence of a variant allele at the one or more sites is predictive that the individual is responsive to bisphosphonate treatment. The variant allele may alter (i.e. reduce or increase) FDPS expression or activity in the individual relative to the wild-type allele, or may be in linkage disequilibrium with a variant allele which alters FDPS expression or activity in the individual.

A site of polymorphism may be in FDPS gene locus or in the genomic region surrounding the FDPS gene, for example in the region between positions 151983001 and 152252001 of chromosome 1. The presence of variant alleles may be determined at one, two, three, four or five or more sites of polymorphism within this region. For example, a site of polymorphism may be a SNP shown in Table 3, or, more preferably, a SNP shown in Table 4.

A site of polymorphism may be in the FDPS gene, for example in the coding region of the FDPS gene or in a non-coding region of the FDPS gene, such as an upstream (5′), intronic or downstream (3′) region. The presence of variant alleles may be determined at one, two, three, four or five or more sites of polymorphism. For example, a site of polymorphism may be a SNP as shown in Table 1.

Variant alleles may include deletions, insertions or substitutions of one or more nucleotides, for example relative to a reference nucleotide sequence (e.g. the sequence of the FDPS genomic region which is set out in gi|51458934 NT_{—}004487.17 or gi|51460383 NT_{—}086596.1). For example a variant allele may be an allele of a single nucleotide polymorphism (SNP), small insertion/deletion polymorphism or variable number tandem repeat (VNTR). Preferably, the variant allele is an allele of a single nucleotide polymorphism. Examples of sites of single nucleotide polymorphism at which a variant allele may be present are shown in Tables 1, 3 and 4.

Methods of the invention may comprise determining, in the sample of nucleic acid obtained from the individual, the presence or absence of a variant allele (for example A, T, G, or C) at a site of polymorphism in the genomic region of the FDPS gene, such as a SNP. More preferably, the presence or absence of the variant allele at the site may be determined in both copies of the region in the genome of the individual. The presence of the variant allele at the site in one or both copies of the genomic region of the FDPS gene may be indicative that the individual has a bone disorder which is responsive to treatment with bisphosphonate.

In some preferred embodiments, the presence or absence of a variant allele, such as a T residue, at dbSNP refSNP ID: NCBI|rs2297480 or a variant allele which shows linkage disequilibrium therewith, may be determined.

refSNP ID: NCBI|rs2297480 is located 91 base pairs upstream of intron 1 of the FDPS gene (position 5769837 in contig gi|51458934 NT_{—}004487.17) or 5386725 in contig gi|51460383 NT_{—}086596 and consists of a G/T polymorphism (note that NCBI dbSNP refers to the complementary strand A/C). NCBI|rs2297480 and its flanking sequences are shown in Table 2.

A variant allele which shows linkage disequilibrium with a variant allele at NCBI|rs2297480, for example a T allele, may be an allele at a site of polymorphism in proximity to NCBI|rs2297480 in the FDPS genetic sequence. For example, the presence of an allelic variant may be determined at one or more sites of polymorphism selected from the group consisting of NCBI|rs16836819, NCBI|rs11556436, NCBI|rs11264358 and NCBI|rs12129895 or other SNP shown in Table 1, or an allelic variant may be determined at one or more sites of polymorphism shown in Table 4 or Table 3.

A method described herein may comprise determining the presence or absence of a T at SNP rs2297480 in the genomic nucleic acid sample obtained from the individual. More preferably, the presence or absence of a T at SNP rs2297480 may be determined in both copies of the FDPS gene in the genome of the individual. The presence of a T residue at SNP rs2297480 in both copies of the FDPS gene (i.e. a TT genotype at rs2297480) is indicative that the individual has a bone disorder which is responsive to treatment with bisphosphonate.

The sample obtained from the individual may be any sample which comprises nucleic acid, preferably genomic nucleic acid, for example a tissue or cell sample, such as a biopsy, or a biological fluid sample, such as a blood sample or a swab.

The presence of a variant allele at one or more sites of polymorphism in the genomic region of the FDPS gene (i.e. the genotype of the individual) may be determined by detecting the presence of a FDPS nucleic acid sequence which comprises the one or more variant alleles in a nucleic acid sample obtained from an individual.

The presence of a variant allele at the one or more sites of polymorphism may be determined by any convenient technique, including amplification of all or part of the genomic region of the FDPS gene, including the FDPS gene itself, sequencing all or part of the genomic region of the FDPS gene, including the FDPS gene itself, and/or hybridisation of a probe which is specific for a variant allele.

A specific amplification reaction such as PCR using one or more pairs of primers may conveniently be employed to amplify all or part of the genomic region of the FDPS gene, including the FDPS gene itself, for example, the portion of the sequence containing or suspected of containing the one or more sites of polymorphism.

In some embodiments, the amplification may be allelic variant specific, such that the presence or absence of amplification product is indicative of the presence of a variation in the FDPS gene of the individual. In other embodiments, the amplified nucleic acid may be sequenced as above, and/or tested in any other way to determine the presence or absence of an allelic variant at the one or more sites of polymorphism.

Suitable amplification reactions include the polymerase chain reaction (PCR). PCR comprises repeated cycles of denaturation of template nucleic acid, annealing of primers to template, and elongation of the primers along the template. PCR is well-known in the art and is described for example in “PCR protocols; A Guide to Methods and Applications”, Eds. Innis et al, 1990, Academic Press, New York, Mullis et al, Cold Spring Harbor Symp. Quant. Biol., 51:263, (1987), Ehrlich (ed), PCR technology, Stockton Press, NY, 1989, and Ehrlich et al, Science, 252:1643-1650, (1991)). The number of cycles, the respective conditions of the individual steps, the composition of reagents within the reaction tube, or any other parameter of the reaction set-up may be varied or adjusted by the skilled person, depending on the circumstances. Additional steps (such as initial denaturing, hot-start, touchdown, enzyme time release PCR, replicative PCR) may also be employed.

Numerous variations and modifications of PCR are known in the art and may be employed by the skilled person in performing the present methods. Chemicals, kits, materials and reagents are commercially available to perform PCR reactions.

Other specific nucleic acid amplification techniques include strand displacement activation, the QB replicase system, the repair chain reaction, the ligase chain reaction, ligation activated transcription, SDA (strand displacement amplification) and TMA (transcription mediated amplification). For convenience, and because it is generally preferred, the term PCR is used herein in contexts where other nucleic acid amplification techniques may be applied by those skilled in the art. Unless the context requires otherwise, reference to PCR should be taken to cover use of any suitable nucleic amplification reaction available in the art.

In some embodiments, the binding of a probe to genomic nucleic acid in the sample, or amplification products thereof, may be determined. The probe may comprise a nucleotide sequence which binds specifically to a nucleic acid sequence which contains a variant allele at one or more sites of polymorphism and does not bind specifically to the nucleic acid sequence which does not contain the variant allele at the one or more polymorphic sites. For example, the probe may bind specifically to the nucleic acid sequence of Table 2 which contains a T at SNP rs2297480 and not bind to the nucleic acid sequence of Table 2 which contains a G at SNP rs2297480. The oligonucleotide probe may comprise a label and binding of the probe may be determined by detecting the presence of the label.

One or more (e.g. two) oligonucleotide probes or primers may be hybridised to the FDPS gene in the sample nucleic acid. Hybridisation will generally be preceded by denaturation to produce single-stranded DNA. The hybridisation may be part of amplification procedure such as PCR, or may be part of a probing procedure not involving amplification. An example procedure would be a combination of PCR and low stringency hybridisation.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include examination of restriction fragment length polymorphisms, amplification using PCR, RN'ase cleavage and allele specific oligonucleotide probing. Probing may employ the standard Southern blotting technique. For instance, DNA may be extracted from cells and digested with different restriction enzymes. Restriction fragments may then be separated by electrophoresis on an agarose gel, before denaturation and transfer to a nitrocellulose filter. Labelled probe may be hybridised to the DNA fragments on the filter and binding determined.

Those skilled in the art are well-able to employ suitable conditions of the desired stringency for selective hybridisation, taking into account factors such as oligonucleotide length and base composition, temperature and so on. Suitable selective hybridisation conditions for oligonucleotides of 17 to 30 bases include hybridization overnight at 42° C. in 6×SSC and washing in 6×SSC at a series of increasing temperatures from 42° C. to 65° C.

Other suitable conditions and protocols are described in Molecular Cloning: a Laboratory Manual: 3rd edition, Sambrook & Russell (2001) Cold Spring Harbor Laboratory Press NY and Current Protocols in Molecular Biology, Ausubel et al. eds. John Wiley & Sons (1992).

In some embodiments, genomic nucleic acid may be analysed using a nucleic acid array.

A nucleic acid array comprises a population of nucleic acid sequences immobilised on a support. Each sequence in the population has a particular defined position on the support. Nucleic acid arrays are well known in the art and may be produced in a number of ways. For example, the nucleic acid sequence may be amplified using the polymerase chain reaction from a cell or library of sequences, or synthesized ex situ using an oligonucleotide synthesis device, and subsequently deposited using a microarraying apparatus. Alternatively, the nucleic acid sequence may be synthesized in situ on the microarray using a method such as piezoelectric deposition of nucleotides.

The number of sequences deposited on the array generally may vary upwards from a minimum of at least 10, 100, 1000, or 10,000 to between 10,000 and several million depending on the technology employed.

In some embodiments, the nucleic acid array is a genomic array comprising a population of genomic sequences from an individual having a bone disorder. In particular, a genomic tiling path array that covers the FDPS gene locus may be employed. In a tiling array, every immobilised nucleic acid, typically each the same size, corresponds to a specific genomic region, with different immobilised nucleic acids containing nucleotide sequences corresponding to shifts of one or more nucleotides relative to each other along the genomic region. For example, a tiling array may be designed such that each nucleic acid from a stretch of genomic sequence that is on the array differs from its adjacent nucleic acid by a shift of a single base pair, so that a series of nucleic acids will represent a moving window across the stretch of genomic sequence. Thus, an array may comprise overlapping immobilised nucleic acid sequences with as little as one nucleotide shifts and as large as the entire size of the nucleic acid, as well as non-overlapping nucleic acids.

Genomic sequences immobilised on an array may be hybridised with a labelled oligonucleotide probe using standard techniques.

In other embodiments, the nucleic acid array may comprise a population of oligonucleotide sequences which correspond to variant alleles at sites of polymorphism in the genome, in particular oligonucleotide sequences which correspond to allelic variants at sites of polymorphism in the FDPS gene locus. The immobilised oligonucleotide probes may then be hybridised with labelled genomic nucleic acid, for example restriction fragments or amplification products, comprising the all or part of the FDPS gene locus from an individual with a bone disorder.

The nucleic acid sequences on the array to which a labelled probe or nucleic acid hybridises may be determined, for example by measuring and recording the label intensity at each position in the array, for example, using an automated DNA microarray reader.

These sequences correspond to the sequence which is present at the site of polymorphism in the individual, and allow the presence of the allelic variant at the site of polymorphism to be determined.

Nucleic acid or an amplified region thereof may be sequenced to identify or determine the presence of an allelic variant at one or more sites of polymorphism in the genomic region of the FDPS gene. An allelic variant may be identified by comparing the sequence obtained with a reference genomic sequence, as described above.

Sequencing may be performed using any one of a range of standard techniques. Sequencing of an amplified product may, for example, involve precipitation with isopropanol, resuspension and sequencing using a TaqFS+ Dye terminator sequencing kit. Extension products may be electrophoresed on an ABI 377 DNA sequencer and data analysed using Sequence Navigator software.

Having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

A bone disorder as described herein is a condition associated with demineralisation or loss of bone density and/or bone quality, including, for example, osteoporosis, glucocorticoid induced osteoporosis, osteitis deformans (“Paget's disease of bone”), bone metastasis (with or without hypercalcemia), multiple myeloma and risk of bone fracture in an individual which is independent of a diagnosis of osteoporosis.

In some preferred embodiments, the bone disorder is osteoporosis, which is a metabolic bone disease characterized by low bone mass and microarchitectural deterioration of bony tissue leading to enhanced bone fragility and a consequent increase in fracture risk. Osteoporosis may be associated with aging, particularly in post-menopausal women, and also certain conditions such as paralysis, or prolonged use of corticosteroids and other drugs.

Bone disorders such as osteoporosis may be generally diagnosed clinically by measurement of bone mineral density (BMD) using dual x-ray absorptiometry (DXA). BMD (g/cm²) is generally described in terms of the number of standard deviations (SDs) from the young normal mean (T score). For example, a T score of less than −1.0 is generally defined as osteopenic and a T score of less than −2.5 is generally defined as osteoporotic. An individual having a bone disorder as described herein may have a T score of less than −1.0, less than −1.5, less then −2 or less than −2.5.

The methods described herein may be useful in predicting the responsiveness of an individual with a bone disorder such as osteoporosis to treatment with a bisphosphonate. Individuals with a high probability of a positive response to treatment with a bisphosphonate may be identified. A positive response may include stabilised or increased bone density or a reduced rate of decrease in bone density. Individuals identified as responsive to bisphosphonates may be treated with a bisphosphonate i.e. bisphosphonate may be administered to an individual identified by the present methods as responsive.

The methods described herein may also be useful to identify individuals with a low probability of a positive response i.e. individuals who are unlikely to respond to treatment with bisphosphonates. Individuals identified as non-responsive to bisphosphonates may not be treated with bisphosphonate, thereby avoiding unnecessary risk of suffering side-effects associated with such treatment, and may undergo a course of treatment with other anti-osteoporosis therapies, for example anabolic agents such as teriparatide and strontium.

Bisphosphonates (also called: diphosphonates) are a class of pyrophosphate analogues that inhibit the resorption of bone and are commonly used in the prevention and treatment of bone disorders characterised by bone fragility, such as osteoporosis, osteitis deformans (“Paget's disease of bone”), bone metastasis (with or without hypercalcemia), and multiple myeloma. Examples of bisphosphonates currently in use as pharmaceuticals include alendronate, clodronate, ibandronate, pamidronate, risedronate and zoledronate.

The methods described herein may also be useful in the selection of patients for clinical trials of candidate compounds for the treatment of bone disorders, for example anti-resorptive and/or anabolic agents for bone turnover.

A method of identifying a cohort of individuals for use in testing candidate anti-resorptive and/or bone anabolic compounds, for example compounds useful in the treatment of bone disorders, may comprise,
identifying a population of individuals having a bone disorder,
determining, in a genomic sample obtained from each of the individuals in said population, the presence or absence of a variant allele at one or more sites of polymorphism in the region of the farnesyl diphosphate synthase (FDPS) gene as described herein,
identifying a cohort of individuals within the population who have a variant allele at one or more sites of polymorphism in the region of the farnesyl diphosphate synthase (FDPS) gene.

The identified cohort may be useful in testing candidate anti-resorptive and/or bone anabolic agent, including pyrophosphate analogues such as bisphosphonates. For example, a candidate compound may be administered to the cohort of individuals and the effect of the compound on the individuals determined.

The presence of a beneficial effect on the cohort of individuals may be indicative that the compound is useful in treating individuals having a bone disorder who have a variant allele at one or more sites of polymorphism in the FDPS gene.

The methods described herein may also be useful in the analysis and stratification of the results of clinical trials of compounds for the treatment of bone disorders, for example anti-resorptive and/or anabolic agents for bone turnover.

Another aspect of the invention provides a method of identifying an anti-resorptive and/or bone anabolic compound, which may, for example be useful in the treatment of a bone disorder, comprising;
treating a population of individuals with a candidate anti-resorptive and/or bone anabolic compound,
determining in a genomic sample obtained from each of the individuals in said population, the presence or absence of a variant allele at one or more sites of polymorphism in the genomic region of the farnesyl diphosphate synthase (FDPS) gene as described herein,
identifying a cohort of individuals within the population who have a variant allele at one or more sites of polymorphism in the genomic region of the farnesyl diphosphate synthase (FDPS) gene,
determining the responsiveness of the individuals in said cohort to the candidate compound.

Another aspect of the invention provides a method of identifying an allelic variant which is associated with the responsiveness of a bone disorder to bisphosphonate comprising;
providing a population of patients having a bone disorder and undergoing treatment with bisphosphonate,
identifying a first cohort of patients in said population who are responsive to bisphosphonate and a second cohort who are unresponsive to bisphosphonate,
determining the presence of allelic variants in the genomic region of the FDPS gene in said first and second cohorts as described herein,
wherein allelic variants present or occurring predominantly in the first but not the second cohort are candidate variants for association with response to bisphosphonate.

A allelic variant may be at a site of polymorphism in the FDPS gene, for example a SNP shown in Table 1, or at a site of polymorphism in the genomic region surrounding the FDPS gene, for example a SNP shown in Table 3 or more preferably a SNP shown in Table 4.

Other aspects of the invention relate to the treatment of bone disorders in individuals having a variant allele at one or more sites of polymorphism in the FDPS gene.

A method of treatment of a bone disorder in an individual having a variant allele at one or more sites of polymorphism in the genomic region of the FDPS gene may comprise:
administering a bisphosphonate to an individual in need thereof.

A bisphosphonate may be used in the manufacture of a medicament for use in the treatment of an individual having a bone disorder,
wherein said individual has a variant allele at one or more sites of polymorphism in the genomic region of the FDPS gene.

A bisphosphonate may be used in the treatment of an individual having a bone disorder,
wherein said individual has a variant allele at one or more sites of polymorphism in the genomic region of the FDPS gene.

Suitable variant alleles are described in more detail above. Preferably, the individual has a variant allele at SNP rs2297480, such as a T allele, or a variant allele in linkage disequilibrium therewith. In some embodiments, the individual may have a TT genotype at SNP rs2297480.

Treatment of a bone disorder may comprise determining the presence of a variant allele at one or more sites of polymorphism in the genomic region of the FDPS gene in said individual. In other words, the genotype of the individual at the one or more sites of polymorphism may be determined. Techniques for determining the presence of a variant allele are described above.

Bone disorders and suitable bisphosphonates are also described in more detail above.

While it is possible for the bisphosphonate to be administered alone, it is preferable to present it as a pharmaceutical composition (e.g., formulation) comprising bisphosphonate, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Pharmaceutical compositions comprising bisphosphonate, for example bisphosphonate admixed together with one or more pharmaceutically acceptable carriers, excipients, buffers, adjuvants, stabilisers, or other materials, as described herein, may be used in the methods described herein. Suitable pharmaceutical compositions comprising bisphosphonate are well known in the art.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The bisphosphonate or pharmaceutical composition comprising the bisphosphonate may be administered to a subject by any suitable route of administration, whether systemically/peripherally or at the site of desired action, including but not limited to, oral (e.g. by ingestion); topical (including e.g. transdermal, parenteral, for example, by injection, including, intravenous Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 mg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the bisphosphonate is in the range of about 100 μg to about 150 mg per month, per two months or per three months. Where the active compound is a salt, an ester, prodrug, or the like, the amount administered is calculated on the basis of the parent compound and so the actual weight to be used is increased proportionately.

Other aspects of the invention relate to kits for identifying an individual having a bone disorder which is responsive to bisphosphonate, or predicting the responsiveness of an individual with a bone disorder to treatment with a bisphosphonate, for example using a method described above.

A kit for identifying an individual having a bone disorder which is responsive to bisphosphonate, or predicting the responsiveness of an individual with a bone disorder to treatment with a bisphosphonate may comprise:
reagents for determining in a genomic sample obtained from the individual, the presence or absence of a variant allele at one or more sites of polymorphism in the genomic region of the farnesyl diphosphate synthase (FDPS) gene,
wherein the presence of a variant allele at the one or more sites being indicative that the individual is responsive to bisphosphonate treatment.

Sites of polymorphism in the genomic region of the FDPS gene, for example in the FDPS gene locus or its surrounding region are described in more detail above. In some embodiments, the kit may comprise reagents for determining the presence or absence of a T at SNP rs2297480 in the FDPS gene. As described above, the presence of a TT genotype at SNP rs2297480 is indicative that the individual is responsive to bisphosphonate treatment.

A kit may comprise amplification reagents for amplifying all or part of the FDPS gene from a genomic sample obtained from an individual. Amplification reagents may include buffers, nucleotides, taq or other polymerase and/or one or more oligonucleotide primers which bind specifically to the FDPS and are suitable for amplifying a region of the gene containing one or more sites of polymorphism, such as SNP rs2297480, for example by PCR.

A kit may comprise detection reagents for determining the presence of one or more sequence variations in the genomic region of the FDPS gene of said individual. Detection means may include labelled oligonucleotide probe which binds to an allelic variant at a site of polymorphism in the genomic region of the FDPS gene or labels, for example for labelling amplified nucleic acid products.

A kit may comprise one or more articles and/or reagents for performance of the method, such as means for providing the test sample itself, e.g. a swab for removing cells from the buccal cavity or a syringe for removing a blood sample (such components generally being sterile).

The kit may further comprise instructions for using the kit in accordance with a method described above.

A kit may further comprise control nucleic acid, for example comprising known alleles at one or more sites of polymorphism in the genomic region of the FDPS gene.

Various further aspects and embodiments of the present invention will be apparent to those skilled in the art in view of the present disclosure. All documents mentioned in this specification are incorporated herein by reference in their entirety.

Certain aspects and embodiments of the invention will now be illustrated by way of example and with reference to the tables described below.

Table 1 shows SNPS in the FDPS gene.

Table 2 shows the sequence surrounding SNP #rs2297480.

Table 3 shows SNPs from the dbSNP database which are located in the genomic region of the FDPS gene between the local recombination hotspots at positions 151983001 and 152252001 on chromosome 1 (NCBI NC_{—}000001 created 29 Aug. 2002).

Table 4 shows SNPs from the HapMap release #20 database (January 2006) which are located in the genomic region of the FDPS gene between the local recombination hotspots at positions 151983001 and 152252001 on chromosome 1 (NCBI NC_{—}000001 created 29 Aug. 2002).

### EXPERIMENTS

Genetic samples were obtained from subjects enrolled in an ongoing clinical programme for treatment of OP through the use of regular intravenous Pamidronate (30 mg three monthly).

The first 24 months of treatment in individuals commencing intravenous amino-bisphosphonate therapy sees a pronounced effect due to the contribution of the reduction in the remodelling space and densitometric estimates of projected bone density are described as indirectly correlating to a multi-compartment pharmacokinetic model throughout this period. In these early stages of treatment the sparse densitometric sampling associated with best clinical practice can thus be fitted to a simple linear time trajectory, thereby supporting an inter-individual comparison of therapeutic response.

By such methodology, 53 subjects (each with two densitometric estimates taken in the first 24 month of Pamidronate therapy) were respectively assigned drug response phenotypes according to their display of ongoing demineralisation (‘response failure’) or stable or improving mineralising (‘response success’). This was achieved by comparison of derived annualised rates of change in projected densitometric estimates of total hip mineralisation. Given a coefficient of error of 1% in the dual energy X-ray absorptiometry imaging technique employed, the 95% certitude limit for ‘clinically significant’ ongoing demineralisation (response failure) was maintained at standard thresholds (a densitometric change of −2.7% or greater).

A search was made of dbSNP to identify regions of the FDPS gene containing single nucleotide polymorphisms (SNPs) with high heterozygosity. A region containing exons 2 and 3 and a region containing exon 12 were chosen for investigation because these regions contained known SNPs with the highest heterozygocity. PCR primers were designed to amplify the above regions and, in addition, two primers were designed for DNA sequencing (see primer list below). DNA was extracted from blood samples using standard organic phase methods and PCR amplification and DNA sequencing was carried out using standard protocols (dye-terminator sequencing using an ABI3100 automated DNA sequencer).

| NAME | SEQUENCE |
|---|---|
| FDS_EX2-3_U | CCTCCTTGGGGCGTAACTCA |
| FDS_EX2-3_L | GCCACAGGTGAATGCCACAC |
| FDS_EX2-3_S1 | TTTTGTTCCCTGCGT ATCC |
| FDS-EX12-U | GGAGTAGAGGATGCCTGGTATG |
| FDS-EX12-L | AGGTTACACGATTATTTATTGAGAGC |
| FDS-EX12-S1 | GTGGGTGGCTTTGGAGAT |

A G/T polymorphism was identified 97 bp upstream of intron 1 of the FDPS gene that corresponded to dbSNP ref SNP ID: rs2297480. A chi-squared test showed clear significance for distribution of response phenotype according to such alleles (respectively TT29 vs 2 and TG/GG 12vs10 p<=0.01).

The odds ratio for the >−2.7 cutoff was found to be 12.0833 (95% CI: 2.2961 to 63.5874) and the odds ratio for the pos/neg cutoff, was found to be 3.1818 (95% CI: 1.0192 to 9.9327). This demonstrates the strength of the observed effect.

TABLE 1

| Position in | | | | | | | |
|---|---|---|---|---|---|---|---|
| contig | | | | | | | Amino |
| gi\|51458934 | | | | d/bSNP | Protein | Codon | acid |
| NT_004487.17 | SNP ID NO: # | Heterozygosity | Function | allele | residue | position | position |
| 5769837 | rs2297480 | 0.498 | Untranslated | A/C | | | |
| 5770216 | rs16836819 | 0.014 | synonymous | T | Asn [N] | 3 | 68 |
| 5770254 | rs11556436 | N.D. | nonsynonymous | G | Arg [R] | 2 | 81 |
| 5770519 | rs11264358 | N.D. | intron | C/T | | | |
| 5771830 | rs12129895 | N.D | intron | G/T | | | |
| 5772044 | rs10458626 | N.D. | intron | A/G | | | |
| 5772419 | rs2148136 | N.D. | nonsynonymous | G | Val [V] | 1 | 120 |
| 5772465 | rs11556432 | N.D. | nonsynonymous | T | Leu [L] | 2 | 135 |
| 5772479 | rs11556437 | N.D. | nonsynonymous | T | Ser [S] | 1 | 140 |
| 5773126 | rs11804127 | N.D. | intron | A/C | | | |
| 5773184 | rs11264359 | 0.483 | intron | A/G | | | |
| 5774570 | rs12409362 | N.D. | intron | C/T | | | |
| 5774616 | rs10796941 | N.D | intron | C/T | | | |
| 5774941 | rs11264360 | N.D. | intron | A/T | | | |
| 5776209 | rs10908462 | N.D. | intron | C/T | | | |
| 5776613 | rs10908463 | N.D. | intron | C/T | | | |
| 5776791 | rs11807340 | N.D. | intron | G/T | | | |
| 5777596 | rs17367421 | 0.055 | intron | C/G | | | |
| 5777943 | rs1409140 | 0.488 | intron | C/T | | | |
| 5779900 | rs11264361 | N.D. | intron | G/T | | | |
| 5780717 | rs1050365 | N.D. | synonymous | T | Leu [L] | 1 | 408 |
| 5767816 | Rs12033064 | N.D. | locus | C/T | | | |
| 5767943 | Rs7552559 | N.D. | locus | A/G | | | |
| 5768213 | Rs11337029 | N.D. | locus | —/T | | | |
| 5768318 | Rs6672284 | N.D. | locus | C/T | | | |
| 5768796 | Rs12043597 | N.D. | locus | C/T | | | |
| 5780927 | Rs12407073 | N.D. | locus | G/T | | | |
| 5780942 | Rs16836822 | 0.014 | locus | G/T | | | |

TABLE 2

TGGGGTACTT TACTCTGTAC CGCCTCCTTA CCCAGCCTTG
TGCACGCCAT CTTGAAGGCA CTGAGTTCTA GCCTGTTTAT
TGTAAGTGGT GATTAGTTGG GTCTCAGTCA CCCAGCCATA
CTTTTTTGTT CCCTGCGTAT CCTTCCTGTA ATTGTCCCCA
AGCACATTCC ACAAGAGGGA GGGGCACTCT GGGCTAAGGC
[T/G]
GGGGTGGGAG TTATCTGGGG AGCTGCCACC ATGCCTCTGC
CTTTGGTGCT TGCCCCTGCA GGGAGTGCTT AGTGCCCCCT
CCCTATGCCA CTCCCAGGAT GCCCCTGTCC CGCTGGTTGA
GATCTGTGGG GGTCTTCCTG CTGCCAGCCC CCTACTGGGC
ACCCCGGGAG AGGTGGCTGG GTTCCCTACG GCGGCCCTCC

TABLE 3

| | | Position on |
|---|---|---|
| | SNP ID NO: # | chromosome 1 |
| 1 | rs406141 | 151985250 |
| 2 | rs2066981 | 151985452 |
| 3 | rs2361529 | 151985896 |
| 4 | rs2075571 | 151987179 |
| 5 | rs370545 | 151988463 |
| 6 | rs914615 | 151988965 |
| 7 | rs6700457 | 151989649 |
| 8 | rs16836684 | 151991033 |
| 9 | rs760077 | 151991855 |
| 10 | rs3738808 | 151993539 |
| 11 | rs12065064 | 151993963 |
| 12 | rs2734403 | 151994159 |
| 13 | rs2734402 | 151994227 |
| 14 | rs2734401 | 151994373 |
| 15 | rs2778496 | 151994440 |
| 16 | rs2990221 | 151994469 |
| 17 | rs2974935 | 151994916 |
| 18 | rs2075570 | 151995237 |
| 19 | rs421050 | 151997440 |
| 20 | rs421016 | 151997489 |
| 21 | rs3115534 | 151998060 |
| 22 | rs3115533 | 151998100 |
| 23 | rs3125562 | 151998311 |
| 24 | rs419697 | 151999024 |
| 25 | rs3115532 | 151999435 |
| 26 | rs404065 | 151999507 |
| 27 | rs3125563 | 151999576 |
| 28 | rs3125564 | 151999577 |
| 29 | rs3125565 | 151999583 |
| 30 | rs3125566 | 151999709 |
| 31 | rs409652 | 151999761 |
| 32 | rs28445596 | 151999802 |
| 33 | rs1057941 | 151999815 |
| 34 | rs3768568 | 152000869 |
| 35 | rs3125561 | 152001580 |
| 36 | rs3115531 | 152001581 |
| 37 | rs1064639 | 152001760 |
| 38 | rs1059732 | 152001877 |
| 39 | rs1064635 | 152001884 |
| 40 | rs1064633 | 152001943 |
| 41 | rs3119758 | 152002211 |
| 42 | rs3119759 | 152002278 |
| 43 | rs3115530 | 152002279 |
| 44 | rs3119760 | 152002293 |
| 45 | rs421585 | 152002953 |
| 46 | rs2990220 | 152003327 |
| 47 | rs12120349 | 152005113 |
| 48 | rs497829 | 152006605 |
| 49 | rs4024046 | 152007214 |
| 50 | rs5777942 | 152007215 |
| 51 | rs12028078 | 152007302 |
| 52 | rs16836748 | 152007752 |
| 53 | rs2049805 | 152008053 |
| 54 | rs6677756 | 152008117 |
| 55 | rs2974931 | 152008288 |
| 56 | rs28498909 | 152009136 |
| 57 | rs10591798 | 152009231 |
| 58 | rs2974930 | 152009790 |
| 59 | rs2974929 | 152010341 |
| 60 | rs2990245 | 152010535 |
| 61 | rs3835732 | 152010628 |
| 62 | rs2990246 | 152010675 |
| 63 | rs2990247 | 152010900 |
| 64 | rs11580040 | 152011295 |
| 65 | rs12407919 | 152012388 |
| 66 | rs2990217 | 152012671 |
| 67 | rs11264343 | 152012739 |
| 68 | rs12723761 | 152013397 |
| 69 | rs3768566 | 152014137 |
| 70 | rs4043 | 152014263 |
| 71 | rs1045253 | 152014308 |
| 72 | rs28595322 | 152015066 |
| 73 | rs2778495 | 152015204 |
| 74 | rs10796940 | 152015762 |
| 75 | rs438459 | 152015780 |
| 76 | rs368793 | 152015783 |
| 77 | rs438450 | 152015791 |
| 78 | rs368766 | 152015799 |
| 79 | rs390685 | 152015846 |
| 80 | rs2974926 | 152015910 |
| 81 | rs567950 | 152016132 |
| 82 | rs11264344 | 152016133 |
| 83 | rs2860587 | 152016151 |
| 84 | rs2361530 | 152016378 |
| 85 | rs2361531 | 152016381 |
| 86 | rs2361532 | 152016385 |
| 87 | rs2361533 | 152016395 |
| 88 | rs4024047 | 152016405 |
| 89 | rs4024048 | 152016407 |
| 90 | rs4024049 | 152016413 |
| 91 | rs2142046 | 152016476 |
| 92 | rs2142045 | 152016503 |
| 93 | rs3115535 | 152017051 |
| 94 | rs3916686 | 152017083 |
| 95 | rs3817647 | 152017278 |
| 96 | rs5777943 | 152017604 |
| 97 | rs1057944 | 152017694 |
| 98 | rs28408650 | 152017757 |
| 99 | rs394757 | 152017757 |
| 100 | rs708606 | 152017767 |
| 101 | rs368060 | 152018081 |
| 102 | rs12747811 | 152018151 |
| 103 | rs2974924 | 152018243 |
| 104 | rs426516 | 152018276 |
| 105 | rs28373017 | 152018404 |
| 106 | rs1800473 | 152018404 |
| 107 | rs12752133 | 152018451 |
| 108 | rs1064651 | 152018591 |
| 109 | rs2990223 | 152018730 |
| 110 | rs28559737 | 152018742 |
| 111 | rs11558184 | 152019158 |
| 112 | rs1064648 | 152019230 |
| 113 | rs2230288 | 152019240 |
| 114 | rs1064647 | 152019295 |
| 115 | rs17401379 | 152019295 |
| 116 | rs17401372 | 152019307 |
| 117 | rs9628662 | 152019414 |
| 118 | rs12743554 | 152019786 |
| 119 | rs1057942 | 152020276 |
| 120 | rs708610 | 152020283 |
| 121 | rs762488 | 152020622 |
| 122 | rs2009578 | 152020699 |
| 123 | rs28678003 | 152020806 |
| 124 | rs381737 | 152021005 |
| 125 | rs1064644 | 152021056 |
| 126 | rs381427 | 152021070 |
| 127 | rs381418 | 152021078 |
| 128 | rs364897 | 152021079 |
| 129 | rs2974923 | 152021256 |
| 130 | rs439898 | 152021494 |
| 131 | rs17423233 | 152021494 |
| 132 | rs7416991 | 152021720 |
| 133 | rs2974922 | 152021746 |
| 134 | rs2974921 | 152021793 |
| 135 | rs2990224 | 152021794 |
| 136 | rs28498204 | 152021838 |
| 137 | rs2075569 | 152022433 |
| 138 | rs17405276 | 152022782 |
| 139 | rs16836761 | 152022819 |
| 140 | rs16836764 | 152022820 |
| 141 | rs3205615 | 152022820 |
| 142 | rs1141807 | 152022820 |
| 143 | rs3205614 | 152022834 |
| 144 | rs1064643 | 152022834 |
| 145 | rs1059731 | 152023528 |
| 146 | rs17405269 | 152023563 |
| 147 | rs17405262 | 152023565 |
| 148 | rs2885305 | 152023616 |
| 149 | rs2361534 | 152023643 |
| 150 | rs2361535 | 152023679 |
| 151 | rs2070679 | 152023714 |
| 152 | rs1141801 | 152023978 |
| 153 | rs1064640 | 152023990 |
| 154 | rs1064638 | 152024006 |
| 155 | rs1064637 | 152024076 |
| 156 | rs1064636 | 152024113 |
| 157 | rs12041778 | 152024910 |
| 158 | rs12071934 | 152025173 |
| 159 | rs12072929 | 152025284 |
| 160 | rs3754484 | 152025361 |
| 161 | rs11430678 | 152025497 |
| 162 | rs11264345 | 152026197 |
| 163 | rs2990225 | 152026812 |
| 164 | rs2990226 | 152026814 |
| 165 | rs2990227 | 152026838 |
| 166 | rs10908459 | 152027139 |
| 167 | rs12034326 | 152027546 |
| 168 | rs10668496 | 152027736 |
| 169 | rs2990228 | 152028220 |
| 170 | rs12406363 | 152028335 |
| 171 | rs1158151 | 152029257 |
| 172 | rs2178815 | 152029792 |
| 173 | rs2178814 | 152029793 |
| 174 | rs2974920 | 152030468 |
| 175 | rs2072648 | 152030716 |
| 176 | rs2075568 | 152031241 |
| 177 | rs734073 | 152031438 |
| 178 | rs734074 | 152031519 |
| 179 | rs1807042 | 152031751 |
| 180 | rs2015296 | 152032254 |
| 181 | rs1546818 | 152032280 |
| 182 | rs3065766 | 152032677 |
| 183 | rs3065762 | 152032677 |
| 184 | rs3065758 | 152032677 |
| 185 | rs2361536 | 152032698 |
| 186 | rs7417746 | 152032749 |
| 187 | rs2361537 | 152032887 |
| 188 | rs741756 | 152032941 |
| 189 | rs2361538 | 152033041 |
| 190 | rs2361539 | 152033042 |
| 191 | rs2361540 | 152033043 |
| 192 | rs2990230 | 152034891 |
| 193 | rs2072647 | 152034910 |
| 194 | rs2075567 | 152034917 |
| 195 | rs2974919 | 152034935 |
| 196 | rs2974918 | 152034937 |
| 197 | rs2990231 | 152035194 |
| 198 | rs12748155 | 152035250 |
| 199 | rs12730000 | 152035545 |
| 200 | rs12730005 | 152035556 |
| 201 | rs10908460 | 152035586 |
| 202 | rs11586220 | 152035714 |
| 203 | rs12084530 | 152035726 |
| 204 | rs11264346 | 152036080 |
| 205 | rs2990232 | 152036663 |
| 206 | rs2974917 | 152036664 |
| 207 | rs2974916 | 152036880 |
| 208 | rs2075566 | 152037164 |
| 209 | rs2974915 | 152037196 |
| 210 | rs2242577 | 152037374 |
| 211 | rs12758281 | 152037466 |
| 212 | rs3119761 | 152037806 |
| 213 | rs878436 | 152038058 |
| 214 | rs909108 | 152038098 |
| 215 | rs2361541 | 152038737 |
| 216 | rs3065799 | 152038740 |
| 217 | rs760075 | 152038859 |
| 218 | rs11557755 | 152039100 |
| 219 | rs2974914 | 152039188 |
| 220 | rs2990233 | 152039190 |
| 221 | rs909107 | 152040203 |
| 222 | rs909106 | 152040212 |
| 223 | rs1318328 | 152040224 |
| 224 | rs12402253 | 152040887 |
| 225 | rs12042020 | 152041119 |
| 226 | rs11587245 | 152041170 |
| 227 | rs12408822 | 152041346 |
| 228 | rs12402578 | 152041509 |
| 229 | rs2990234 | 152041828 |
| 230 | rs2974913 | 152041830 |
| 231 | rs4971068 | 152041936 |
| 232 | rs9729564 | 152042295 |
| 233 | rs1548224 | 152042459 |
| 234 | rs11264347 | 152042839 |
| 235 | rs11264348 | 152042988 |
| 236 | rs3125560 | 152043010 |
| 237 | rs17857819 | 152043204 |
| 238 | rs17845043 | 152043204 |
| 239 | rs3180018 | 152043204 |
| 240 | rs11557754 | 152043450 |
| 241 | rs1076555 | 152043527 |
| 242 | rs760074 | 152043578 |
| 243 | rs12084873 | 152043640 |
| 244 | rs2990235 | 152043789 |
| 245 | rs2361542 | 152043971 |
| 246 | rs760073 | 152044552 |
| 247 | rs2242576 | 152044618 |
| 248 | rs1046188 | 152045147 |
| 249 | rs2990236 | 152045385 |
| 250 | rs3887 | 152045841 |
| 251 | rs1049090 | 152046188 |
| 252 | rs2990237 | 152046443 |
| 253 | rs2990238 | 152046547 |
| 254 | rs2990239 | 152046643 |
| 255 | rs1076556 | 152047894 |
| 256 | rs12044394 | 152047968 |
| 257 | rs12043655 | 152047984 |
| 258 | rs760081 | 152048075 |
| 259 | rs7355033 | 152048213 |
| 260 | rs2008420 | 152048312 |
| 261 | rs7417380 | 152048456 |
| 262 | rs4543784 | 152048456 |
| 263 | rs3065804 | 152048469 |
| 264 | rs2008404 | 152048471 |
| 265 | rs1078699 | 152048566 |
| 266 | rs760078 | 152048608 |
| 267 | rs2974936 | 152048949 |
| 268 | rs6696982 | 152048998 |
| 269 | rs11411609 | 152049037 |
| 270 | rs11389873 | 152049038 |
| 271 | rs11428813 | 152049040 |
| 272 | rs2990241 | 152049786 |
| 273 | rs2990242 | 152049955 |
| 274 | rs4971069 | 152050130 |
| 275 | rs4971070 | 152050277 |
| 276 | rs4971071 | 152050742 |
| 277 | rs2990243 | 152050812 |
| 278 | rs2990244 | 152050814 |
| 279 | rs2049804 | 152050855 |
| 280 | rs2885306 | 152051680 |
| 281 | rs2049803 | 152051683 |
| 282 | rs2049802 | 152051685 |
| 283 | rs2049801 | 152051691 |
| 284 | rs2049800 | 152051693 |
| 285 | rs2361543 | 152052697 |
| 286 | rs9427191 | 152053066 |
| 287 | rs11577338 | 152053150 |
| 288 | rs489016 | 152053538 |
| 289 | rs12749700 | 152053787 |
| 290 | rs28463199 | 152054176 |
| 291 | rs2236863 | 152054434 |
| 292 | rs12563994 | 152057165 |
| 293 | rs12732972 | 152057756 |
| 294 | rs12732984 | 152057773 |
| 295 | rs10128085 | 152058391 |
| 296 | rs12749306 | 152060780 |
| 297 | rs28693067 | 152061120 |
| 298 | rs11264349 | 152061186 |
| 299 | rs7549276 | 152061648 |
| 300 | rs11264350 | 152061913 |
| 301 | rs4269766 | 152061981 |
| 302 | rs7551854 | 152062070 |
| 303 | rs7554780 | 152062771 |
| 304 | rs12044063 | 152063764 |
| 305 | rs7367998 | 152064323 |
| 306 | rs12049375 | 152065906 |
| 307 | rs7543234 | 152066381 |
| 308 | rs11264351 | 152066568 |
| 309 | rs7520184 | 152066656 |
| 310 | rs12239421 | 152068441 |
| 311 | rs11264352 | 152068910 |
| 312 | rs11264353 | 152068979 |
| 313 | rs11264354 | 152069155 |
| 314 | rs12724449 | 152069304 |
| 315 | rs12402606 | 152070055 |
| 316 | rs11264355 | 152070565 |
| 317 | rs12118947 | 152071521 |
| 318 | rs3814319 | 152071825 |
| 319 | rs3814318 | 152071829 |
| 320 | rs8177998 | 152071925 |
| 321 | rs8177997 | 152072092 |
| 322 | rs8177996 | 152072098 |
| 323 | rs8847 | 152072396 |
| 324 | rs8177995 | 152072971 |
| 325 | rs932972 | 152073169 |
| 326 | rs8177994 | 152073422 |
| 327 | rs1052177 | 152073423 |
| 328 | rs17858737 | 152073456 |
| 329 | rs17845777 | 152073456 |
| 330 | rs1052176 | 152073456 |
| 331 | rs10565071 | 152073895 |
| 332 | rs8177993 | 152074167 |
| 333 | rs8177992 | 152074229 |
| 334 | rs8177991 | 152074276 |
| 335 | rs8177990 | 152074308 |
| 336 | rs8177989 | 152074353 |
| 337 | rs3020786 | 152074422 |
| 338 | rs8177988 | 152074722 |
| 339 | rs3762272 | 152074850 |
| 340 | rs8177987 | 152075087 |
| 341 | rs11264356 | 152075116 |
| 342 | rs3020785 | 152075124 |
| 343 | rs8177986 | 152075362 |
| 344 | rs8177985 | 152075487 |
| 345 | rs8177984 | 152075676 |
| 346 | rs4620533 | 152075686 |
| 347 | rs8177983 | 152075712 |
| 348 | rs8177982 | 152076259 |
| 349 | rs8177981 | 152076457 |
| 350 | rs8177980 | 152076483 |
| 351 | rs8177979 | 152076503 |
| 352 | rs8177978 | 152076866 |
| 353 | rs8177977 | 152076911 |
| 354 | rs8177976 | 152076945 |
| 355 | rs8177975 | 152077073 |
| 356 | rs8177974 | 152077243 |
| 357 | rs8177973 | 152077696 |
| 358 | rs2071053 | 152078250 |
| 359 | rs8177972 | 152078265 |
| 360 | rs8177971 | 152078519 |
| 361 | rs3020784 | 152078718 |
| 362 | rs2990219 | 152078720 |
| 363 | rs8177970 | 152078734 |
| 364 | rs3020782 | 152079082 |
| 365 | rs2990218 | 152079084 |
| 366 | rs8177969 | 152079111 |
| 367 | rs8177968 | 152079408 |
| 368 | rs8177967 | 152079586 |
| 369 | rs8177966 | 152079647 |
| 370 | rs8177965 | 152079687 |
| 371 | rs12067675 | 152081193 |
| 372 | rs12741350 | 152081498 |
| 373 | rs11802924 | 152081913 |
| 374 | rs11264357 | 152082031 |
| 375 | rs12032821 | 152082396 |
| 376 | rs12040032 | 152082534 |
| 377 | rs3020781 | 152082849 |
| 378 | rs8177964 | 152082853 |
| 379 | rs8177963 | 152082903 |
| 380 | rs8177962 | 152083064 |
| 381 | rs8177961 | 152083237 |
| 382 | rs3020783 | 152084094 |
| 383 | rs8177960 | 152084407 |
| 384 | rs7524950 | 152085347 |
| 385 | rs12037847 | 152085624 |
| 386 | rs4971072 | 152086942 |
| 387 | rs12726199 | 152087326 |
| 388 | rs12032720 | 152088033 |
| 389 | rs7534795 | 152088626 |
| 390 | rs10630800 | 152088660 |
| 391 | rs10908461 | 152088800 |
| 392 | rs1888929 | 152089247 |
| 393 | rs3834761 | 152089456 |
| 394 | rs12033064 | 152090534 |
| 395 | rs7552559 | 152090661 |
| 396 | rs11337029 | 152090931 |
| 397 | rs6672284 | 152091036 |
| 398 | rs12043597 | 152091514 |
| 399 | rs2297480 | 152092555 |
| 400 | rs16836819 | 152092934 |
| 401 | rs11556436 | 152092972 |
| 402 | rs11264358 | 152093237 |
| 403 | rs12129895 | 152094548 |
| 404 | rs10458626 | 152094762 |
| 405 | rs2148136 | 152095137 |
| 406 | rs11556432 | 152095183 |
| 407 | rs11556437 | 152095197 |
| 408 | rs11804127 | 152095844 |
| 409 | rs11264359 | 152095902 |
| 410 | rs12409362 | 152097288 |
| 411 | rs10796941 | 152097334 |
| 412 | rs11264360 | 152097659 |
| 413 | rs10908462 | 152098927 |
| 414 | rs10908463 | 152099331 |
| 415 | rs11807340 | 152099509 |
| 416 | rs17367421 | 152100314 |
| 417 | rs1409140 | 152100661 |
| 418 | rs11264361 | 152102618 |
| 419 | rs1050365 | 152103435 |
| 420 | rs12407073 | 152103645 |
| 421 | rs16836822 | 152103660 |
| 422 | rs12741581 | 152104504 |
| 423 | rs4971074 | 152104605 |
| 424 | rs11589917 | 152106085 |
| 425 | rs874870 | 152106821 |
| 426 | rs914616 | 152107347 |
| 427 | rs4971075 | 152107845 |
| 428 | rs12087231 | 152108076 |
| 429 | rs12061020 | 152111091 |
| 430 | rs9427215 | 152112249 |
| 431 | rs12745819 | 152112766 |
| 432 | rs12728412 | 152112818 |
| 433 | rs9803672 | 152114867 |
| 434 | rs11294228 | 152115307 |
| 435 | rs7546549 | 152115324 |
| 436 | rs10530618 | 152115349 |
| 437 | rs11414431 | 152115713 |
| 438 | rs7549232 | 152115796 |
| 439 | rs6692183 | 152117400 |
| 440 | rs6677385 | 152117654 |
| 441 | rs6695298 | 152118132 |
| 442 | rs11552268 | 152118363 |
| 443 | rs1047304 | 152118750 |
| 444 | rs16836837 | 152121000 |
| 445 | rs12748814 | 152123516 |
| 446 | rs11362270 | 152123657 |
| 447 | rs28417969 | 152124243 |
| 448 | rs4644482 | 152124554 |
| 449 | rs4971050 | 152125102 |
| 450 | rs4971051 | 152126120 |
| 451 | rs10477032 | 152126124 |
| 452 | rs6690002 | 152126641 |
| 453 | rs6671191 | 152128002 |
| 454 | rs11417303 | 152128051 |
| 455 | rs11264362 | 152128688 |
| 456 | rs12562734 | 152130016 |
| 457 | rs10157264 | 152130184 |
| 458 | rs3748558 | 152130787 |
| 459 | rs11264363 | 152131381 |
| 460 | rs28533380 | 152131761 |
| 461 | rs28718212 | 152131763 |
| 462 | rs28625826 | 152131777 |
| 463 | rs28491236 | 152131811 |
| 464 | rs12131079 | 152132741 |
| 465 | rs6659005 | 152132955 |
| 466 | rs6670530 | 152133351 |
| 467 | rs6670726 | 152133505 |
| 468 | rs12049455 | 152133565 |
| 469 | rs12735478 | 152133853 |
| 470 | rs12756406 | 152133855 |
| 471 | rs12024696 | 152134622 |
| 472 | rs12021631 | 152134751 |
| 473 | rs7523189 | 152134755 |
| 474 | rs12239114 | 152135215 |
| 475 | rs28465679 | 152135605 |
| 476 | rs6672663 | 152136589 |
| 477 | rs6682261 | 152138803 |
| 478 | rs11264364 | 152139997 |
| 479 | rs12029944 | 152140076 |
| 480 | rs4472748 | 152141204 |
| 481 | rs28753710 | 152141230 |
| 482 | rs7415003 | 152141469 |
| 483 | rs4492610 | 152141469 |
| 484 | rs12748121 | 152144411 |
| 485 | rs12753466 | 152145066 |
| 486 | rs11264365 | 152146067 |
| 487 | rs7340058 | 152148006 |
| 488 | rs7339988 | 152148103 |
| 489 | rs7340071 | 152148127 |
| 490 | rs11581222 | 152148260 |
| 491 | rs11264366 | 152148484 |
| 492 | rs6683631 | 152149097 |
| 493 | rs12755518 | 152150068 |
| 494 | rs12738013 | 152150078 |
| 495 | rs12742159 | 152150101 |
| 496 | rs12755543 | 152150103 |
| 497 | rs12746371 | 152150366 |
| 498 | rs12746379 | 152150374 |
| 499 | rs11264367 | 152150411 |
| 500 | rs12128553 | 152150678 |
| 501 | rs12565122 | 152151286 |
| 502 | rs12087625 | 152155264 |
| 503 | rs6656587 | 152157037 |
| 504 | rs5777944 | 152157865 |
| 505 | rs5005770 | 152158116 |
| 506 | rs12035771 | 152158714 |
| 507 | rs28438500 | 152159644 |
| 508 | rs12078402 | 152159762 |
| 509 | rs12048260 | 152161354 |
| 510 | rs12041057 | 152161459 |
| 511 | rs959485 | 152162311 |
| 512 | rs4622056 | 152162466 |
| 513 | rs12134842 | 152162614 |
| 514 | rs4601578 | 152162619 |
| 515 | rs12754454 | 152167354 |
| 516 | rs12729287 | 152167355 |
| 517 | rs12754459 | 152167363 |
| 518 | rs12750270 | 152167364 |
| 519 | rs6659913 | 152168624 |
| 520 | rs11588849 | 152168737 |
| 521 | rs6665823 | 152169437 |
| 522 | rs7554397 | 152169900 |
| 523 | rs12406331 | 152170109 |
| 524 | rs2025669 | 152170682 |
| 525 | rs7541017 | 152171122 |
| 526 | rs10672932 | 152171586 |
| 527 | rs11586577 | 152171685 |
| 528 | rs12081067 | 152172055 |
| 529 | rs12041011 | 152172073 |
| 530 | rs11264368 | 152172358 |
| 531 | rs7416976 | 152172523 |
| 532 | rs11264369 | 152172903 |
| 533 | rs6694257 | 152172953 |
| 534 | rs6669502 | 152173145 |
| 535 | rs6661389 | 152173509 |
| 536 | rs6670014 | 152173651 |
| 537 | rs7527209 | 152174024 |
| 538 | rs12407344 | 152174755 |
| 539 | rs11585174 | 152176872 |
| 540 | rs12040666 | 152176878 |
| 541 | rs7539746 | 152177758 |
| 542 | rs12748798 | 152179670 |
| 543 | rs12748811 | 152179684 |
| 544 | rs12752987 | 152179698 |
| 545 | rs12748817 | 152179706 |
| 546 | rs12022700 | 152179823 |
| 547 | rs16836847 | 152180744 |
| 548 | rs16836848 | 152180753 |
| 549 | rs7556102 | 152181276 |
| 550 | rs7536194 | 152182278 |
| 551 | rs12025532 | 152182375 |
| 552 | rs12025722 | 152183043 |
| 553 | rs7517139 | 152183088 |
| 554 | rs7542252 | 152183980 |
| 555 | rs11264370 | 152184520 |
| 556 | rs10158037 | 152185683 |
| 557 | rs1886905 | 152185745 |
| 558 | rs10796942 | 152187896 |
| 559 | rs12121568 | 152188901 |
| 560 | rs10158907 | 152190613 |
| 561 | rs10712023 | 152190728 |
| 562 | rs10618305 | 152191813 |
| 563 | rs10908464 | 152194450 |
| 564 | rs12738514 | 152194670 |
| 565 | rs12046473 | 152195499 |
| 566 | rs12093804 | 152195847 |
| 567 | rs11345039 | 152198822 |
| 568 | rs12058261 | 152199263 |
| 569 | rs6665939 | 152199823 |
| 570 | rs12734374 | 152201924 |
| 571 | rs11577694 | 152202413 |
| 572 | rs10908465 | 152202761 |
| 573 | rs11442413 | 152204204 |
| 574 | rs28833498 | 152205504 |
| 575 | rs28791794 | 152206459 |
| 576 | rs28850118 | 152206833 |
| 577 | rs7531890 | 152207843 |
| 578 | rs7517777 | 152207959 |
| 579 | rs11459295 | 152209996 |
| 580 | rs7532714 | 152211169 |
| 581 | rs7522660 | 152211316 |
| 582 | rs9787192 | 152211318 |
| 583 | rs7514174 | 152212365 |
| 584 | rs12025843 | 152214083 |
| 585 | rs12028416 | 152214234 |
| 586 | rs12026638 | 152214333 |
| 587 | rs16836851 | 152215420 |
| 588 | rs12069996 | 152215435 |
| 589 | rs28516425 | 152216112 |
| 590 | rs7355130 | 152216204 |
| 591 | rs12124051 | 152216517 |
| 592 | rs16865367 | 152216921 |
| 593 | rs1061116 | 152217075 |
| 594 | rs12082169 | 152217087 |
| 595 | rs12082171 | 152217107 |
| 596 | rs12082219 | 152217220 |
| 597 | rs12564667 | 152217724 |
| 598 | rs12041534 | 152220169 |
| 599 | rs12138411 | 152220515 |
| 600 | rs4971053 | 152221708 |
| 601 | rs13375379 | 152222434 |
| 602 | rs12070566 | 152223903 |
| 603 | rs7549186 | 152223994 |
| 604 | rs11264371 | 152225110 |
| 605 | rs12086165 | 152225226 |
| 606 | rs10465954 | 152225460 |
| 607 | rs11264372 | 152226003 |
| 608 | rs1325908 | 152226377 |
| 609 | rs7349067 | 152227021 |
| 610 | rs10796943 | 152227802 |
| 611 | rs12730906 | 152227841 |
| 612 | rs12034526 | 152227886 |
| 613 | rs12037824 | 152228254 |
| 614 | rs11588832 | 152228836 |
| 615 | rs10796944 | 152229401 |
| 616 | rs11264373 | 152229476 |
| 617 | rs6668947 | 152230292 |
| 618 | rs10752610 | 152231186 |
| 619 | rs12755007 | 152234057 |
| 620 | rs11264374 | 152234585 |
| 621 | rs12094250 | 152235968 |
| 622 | rs12095569 | 152236692 |
| 623 | rs11264375 | 152237138 |
| 624 | rs10700457 | 152237649 |
| 625 | rs10701330 | 152237650 |
| 626 | rs11264376 | 152239006 |
| 627 | rs6684889 | 152240135 |
| 628 | rs1536255 | 152240223 |
| 629 | rs7536476 | 152240547 |
| 630 | rs10796945 | 152240761 |
| 631 | rs12239100 | 152241549 |
| 632 | rs6658401 | 152242173 |
| 633 | rs10796946 | 152242563 |
| 634 | rs2362361 | 152242772 |
| 635 | rs2362362 | 152242774 |
| 636 | rs10908466 | 152242798 |
| 637 | rs7531517 | 152243500 |
| 638 | rs5777945 | 152243765 |
| 639 | rs7529556 | 152246812 |
| 640 | rs12724079 | 152247015 |
| 641 | rs5777946 | 152248660 |
| 642 | rs12067371 | 152250742 |

TABLE 4

| p | Position on | |
|---|---|---|
| SNP ID NO: # | chromosome 1 | ref_allele |
| rs10158907 | 152190613 | A |
| rs1045253 | 152014308 | C |
| rs1052176 | 152073456 | C |
| rs1057941 | 151999815 | C |
| rs1064640 | 152023990 | A |
| rs10752610 | 152231186 | T |
| rs1076556 | 152047894 | A |
| rs1078699 | 152048566 | A |
| rs10796943 | 152227802 | T |
| rs10908465 | 152202761 | C |
| rs11264345 | 152026197 | T |
| rs11264351 | 152066568 | G |
| rs11264352 | 152068910 | T |
| rs11264355 | 152070565 | C |
| rs11264359 | 152095902 | A |
| rs11264360 | 152097659 | T |
| rs11264361 | 152102618 | T |
| rs11264367 | 152150411 | T |
| rs11264369 | 152172903 | T |
| rs11264371 | 152225110 | C |
| rs11264372 | 152226003 | A |
| rs11264375 | 152237138 | T |
| rs11264376 | 152239006 | T |
| rs11577694 | 152202413 | A |
| rs11585174 | 152176872 | T |
| rs11588849 | 152168737 | C |
| rs11589917 | 152106085 | C |
| rs11807340 | 152099509 | G |
| rs12026638 | 152214333 | T |
| rs12029944 | 152140076 | G |
| rs12032720 | 152088033 | G |
| rs12032821 | 152082396 | G |
| rs12033064 | 152090534 | T |
| rs12035771 | 152158714 | T |
| rs12040666 | 152176878 | G |
| rs12041057 | 152161459 | G |
| rs12041534 | 152220169 | C |
| rs12043597 | 152091514 | C |
| rs12046473 | 152195499 | T |
| rs12048260 | 152161354 | C |
| rs12061020 | 152111091 | C |
| rs12069996 | 152215435 | G |
| rs12070566 | 152223903 | T |
| rs12078402 | 152159762 | A |
| rs12082169 | 152217087 | C |
| rs12082171 | 152217107 | C |
| rs12082219 | 152217220 | C |
| rs12087231 | 152108076 | G |
| rs12093804 | 152195847 | T |
| rs12118947 | 152071521 | A |
| rs12131079 | 152132741 | C |
| rs12134842 | 152162614 | A |
| rs12138411 | 152220515 | C |
| rs12239114 | 152135215 | G |
| rs12239421 | 152068441 | C |
| rs12407073 | 152103645 | G |
| rs12407919 | 152012388 | C |
| rs12724079 | 152247015 | T |
| rs12726199 | 152087326 | A |
| rs12730906 | 152227841 | C |
| rs12738514 | 152194670 | T |
| rs12741581 | 152104504 | C |
| rs12748814 | 152123516 | C |
| rs12750270 | 152167364 | A |
| rs1325908 | 152226377 | G |
| rs13375379 | 152222434 | G |
| rs16836822 | 152103660 | T |
| rs16836851 | 152215420 | A |
| rs17367421 | 152100314 | G |
| rs1886905 | 152185745 | T |
| rs2049805 | 152008053 | A |
| rs2066981 | 151985452 | C |
| rs2236863 | 152054434 | C |
| rs2242577 | 152037374 | G |
| rs2297480 | 152092555 | A |
| rs2362362 | 152242774 | G |
| rs2734403 | 151994159 | T |
| rs2974922 | 152021746 | T |
| rs2990218 | 152079084 | T |
| rs2990219 | 152078720 | C |
| rs2990227 | 152026838 | G |
| rs2990228 | 152028220 | A |
| rs2990245 | 152010535 | C |
| rs2990247 | 152010900 | C |
| rs3020781 | 152082849 | T |
| rs3119759 | 152002278 | A |
| rs3125562 | 151998311 | C |
| rs3180018 | 152043204 | G |
| rs364897 | 152021079 | A |
| rs3738808 | 151993539 | T |
| rs3748558 | 152130787 | C |
| rs3768566 | 152014137 | G |
| rs3768568 | 152000869 | G |
| rs406141 | 151985250 | C |
| rs421585 | 152002953 | A |
| rs4269766 | 152061981 | G |
| rs438450 | 152015791 | C |
| rs4472748 | 152141204 | A |
| rs4601578 | 152162619 | G |
| rs4S20533 | 152075686 | C |
| rs4622056 | 152162466 | T |
| rs4644482 | 152124554 | G |
| rs4971050 | 152125102 | C |
| rs4971051 | 152126120 | T |
| rs4971053 | 152221708 | T |
| rs4971072 | 152086942 | G |
| rs4971074 | 152104605 | T |
| rs5005770 | 152158116 | A |
| rs6659913 | 152168624 | A |
| rs6670530 | 152133351 | G |
| rs6672663 | 152136589 | T |
| rs6684889 | 152240135 | T |
| rs6690002 | 152126641 | C |
| rs6694257 | 152172953 | C |
| rs6695298 | 152118132 | T |
| rs734073 | 152031438 | A |
| rs7355130 | 152216204 | G |
| rs7416991 | 152021720 | T |
| rs7529556 | 152246812 | C |
| rs7531517 | 152243500 | T |
| rs7531890 | 152207843 | T |
| rs7536476 | 152240547 | A |
| rs7539746 | 152177758 | G |
| rs7541017 | 152171122 | C |
| rs7549186 | 152223994 | T |
| rs7549232 | 152115796 | G |
| rs7549276 | 152061648 | G |
| rs7551854 | 152062070 | G |
| rs7556102 | 152181276 | C |
| rs8177960 | 152084407 | C |
| rs8177962 | 152083064 | C |
| rs8177963 | 152082903 | T |
| rs8177967 | 152079586 | G |
| rs8177969 | 152079111 | G |
| rs8177973 | 152077696 | T |
| rs8177974 | 152077243 | T |
| rs8177975 | 152077073 | G |
| rs8177980 | 152076483 | A |
| rs8177981 | 152076457 | C |
| rs8177986 | 152075362 | C |
| rs8177988 | 152074722 | G |
| rs874870 | 152106821 | T |
| rs914615 | 151988965 | A |
| rs932972 | 152073169 | C |
| rs959485 | 152162311 | G |
| rs9628662 | 152019414 | T |

1. A method of identifying an individual having a bone disorder which is responsive to bisphosphonate, or predicting the responsiveness of an individual with a bone disorder to treatment with a bisphosphonate, the method comprising:
determining in a nucleic acid sample obtained from the individual, the presence or absence of a variant allele at one or more sites of polymorphism in the genomic region of the farnesyl diphosphate synthase (FDPS) gene,
the presence of a variant allele at the one or more sites being indicative that the individual is responsive to bisphosphonates.

2. A method according to claim 1 wherein the one or more sites of polymorphism are single nucleotide polymorphisms.

3. A method according to claim 1 or claim 2 wherein the presence or absence of a variant allele is determined at one or more sites of polymorphism in the genomic region between nucleotides 151983001 and 152252001 of chromosome 1.

4. A method according to claim 3 wherein the one or more sites of polymorphism are single nucleotide polymorphisms shown in Table 3.

5. A method according to claim 4 wherein the one or more sites of polymorphism are single nucleotide polymorphisms shown in Table 4.

6. A method according to claim 1 wherein the one or more sites of polymorphism are in the farnesyl diphosphate synthase (FDPS) gene.

7. A method according to claim 6 wherein the one or more sites of polymorphism are shown in Table 1.

8. A method according to claim 1 wherein the presence or absence of a variant allele at SNP rs2297480 or a variant allele in linkage disequilibrium therewith is determined.

9. A method according to claim 8 wherein the presence or absence of a T at SNP rs2297480 is determined.

10. A method according to claim 9 wherein the presence of a T at SNP rs2297480 is indicative that the individual is responsiveness to bisphosphonate.

11. A method according to claim 9 wherein the presence or absence of a T at SNP rs2297480 in both copies of the FDPS gene of said individual is determined.

12. A method according to claim 11 wherein the presence of a TT genotype at SNP rs2297480 is indicative that the individual is responsiveness to bisphosphonate.

13. A method according to claim 1 wherein the presence of a variant allele at the one or more sites of polymorphism is determined by amplification of all or part of the genomic region of the farnesyl diphosphate synthase (FDPS) gene.

14. A method according to claim 1 wherein the presence of a variant allele at the one or more sites of polymorphism is determined by sequencing all or part of the genomic region of the farnesyl diphosphate synthase (FDPS) gene or an amplified portion thereof.

15. A method according to claim 1 the presence of a variant allele at the one or more sites of polymorphism is determined by hybridisation of an allele specific probe to the genomic region of the farnesyl diphosphate synthase (FDPS) gene or an amplified portion thereof.

16. A method according to claim 1 wherein the bone disorder is osteoporosis, glucocorticoid induced osteoporosis, glucocorticoid-induced osteoporosis, osteitis deformans (“Paget's disease of bone”), bone metastasis (with or without hypercalcemia), multiple myeloma or increased risk of bone fracture independent of osteoporosis.

17. A method according to claim 1 wherein the bisphosphonate is selected from the group consisting of alendronate, clodronate, ibandronate, pamidronate, risedronate and zoledronate.

18-24. (canceled)

25. A method of treatment of a bone disorder in an individual having a variant allele at one or more sites of polymorphism in the genomic region of the farnesyl diphosphate synthase (FDPS) gene, the method comprising:
administering a bisphosphonate to an individual in need thereof.

26. A method according to claim 25 wherein the individual has a variant allele at SNP rs2297480 or a variant allele in which is linkage disequilibrium with a variant allele at SNP rs2297480.

27. A method according to claim 26 wherein the individual has a T allele at SNP rs2297480 or a variant allele in which is linkage disequilibrium with a T allele at SNP rs2297480.

28. A method according to claim 27 wherein the individual has the TT genotype at SNP rs2297480.

29. A method according to claim 25 wherein the treatment comprises determining the presence of a variant allele at one or more sites of polymorphism in the genomic region of the FDPS gene in said individual.

30. A method according to claim 25 wherein the bone disorder is osteoporosis, glucocorticoid induced osteoporosis, glucocorticoid-induced osteoporosis, osteitis deformans (“Paget's disease of bone”), bone metastasis (with or without hypercalcemia), multiple myeloma or increased risk of bone fracture independent of osteoporosis.

31. A method according to claim 25 wherein the bisphosphonate is selected from the group consisting of alendronate, clodronate, ibandronate, pamidronate, risedronate and zoledronate.

32. A method of identifying a cohort of individuals for use in testing candidate compounds for the treatment of bone disorders comprising
identifying a population of individuals having a bone disorder,
determining, in a genomic sample obtained from each of the individuals in said population, the presence or absence of a variant allele at one or more sites of polymorphism in the genomic region of the farnesyl diphosphate synthase (FDPS) gene, and,
identifying a cohort of individuals within the population who have a variant allele at one or more sites of polymorphism in the genomic region of the farnesyl diphosphate synthase (FDPS) gene.

33. A method according to claim 32 comprising administering a candidate compound to the cohort of individuals and determining the effect of the compound on the individuals.

34. A method of identifying a compound useful in the treatment of a bone disorder comprising
treating a population of individuals having a bone disorder with a candidate compound,
determining in a genomic sample obtained from each of the individuals in said population, the presence or absence of a variant allele at one or more sites of polymorphism in the genomic region of the farnesyl diphosphate synthase (FDPS) gene
identifying a cohort of individuals within the population who have a variant allele at one or more sites of polymorphism in the genomic region of the farnesyl diphosphate synthase (FDPS) gene,
determining the responsiveness of the individuals in said cohort to the candidate compound.

35. A method of identifying an allelic variant which is associated with the responsiveness of a bone disorder to bisphosphonate comprising;
providing a population of patients having a bone disorder and undergoing treatment with bisphosphonate,
identifying a first cohort of patients in said population who are responsive to bisphosphonate and a second cohort who are unresponsive to bisphosphonate,
determining the presence of allelic variants in the genomic region of the farnesyl diphosphate synthase (FDPS) gene in said first and second cohorts,
wherein allelic variants present or occurring predominantly in the first but not the second cohort are candidate variants for association with response to bisphosphonate.

36. A kit for identifying an individual having a bone disorder which is responsive to bisphosphonate comprising:
reagents for determining the presence or absence of a variant allele at one or more sites of polymorphism in the genomic region of the farnesyl diphosphate synthase (FDPS) gene in a genomic sample obtained from the individual,
wherein the presence of a variant allele at the one or more sites being indicative that the individual is responsive to bisphosphonate treatment.
